(19) United States
(12) Patent Application Publication
(10) Pub. No.: US 2006/0193751 A1
(43) Pub. Date: Aug. 31, 2006

(54) Packing device for chamber sample inlet

(76) Inventors: Sung-ouk Jung, Suwon-si (KR); Soo-suk Lee, Suwon-si (KR); Yu-jin Seo, Siheung-si (KR); Chang-eun Yoo, Seoul (KR); Hun-joo Lee, Seoul (KR)

Correspondence Address: CANTOR COLBURN, LLP, 55 GRIFFIN ROAD SOUTH, BLOOMFIELD, CT, 06002, US

(21) Appl. No.: 11/296,023

(22) Filed: Dec. 6, 2005

(30) Foreign Application Priority Data
Dec. 6, 2004 (KR) 10-2004-0101656

(51) Int. Cl. B01L 3/00; C12M 1/34

(52) U.S. Cl. 422/99; 435/287.2; 436/514

(57) ABSTRACT

A packing device used to pack a sample inlet formed in an upper cover of a chamber is provided. The packing device has an air vent formed in the surface of an upper portion of the sample inlet contacting a cap or in the surface of a lower portion of the cap contacting the sample inlet. The movement of the sample solution in the chamber can be prevented by using a structural design in which air pressure generated due to the volume of a packing cap inserted into the sample inlet after the sample is injected into the chamber through the sample inlet to react with reaction materials, such as biomolecules, is not generated inside of the chamber. In addition, in a hybridization chamber in which a biochip is placed, the spot area on the biochip can be effectively agitated with the sample solution, thus increasing hybridization efficiency.

### CROSS-REFERENCE TO RELATED PATENT APPLICATIONS

This application claims the benefit of Korean Patent Application No. 10-2004-0101656, filed on Dec. 6, 2004, in the Korean Intellectual Property Office, the disclosure of which is incorporated herein in its entirety by reference.

### BACKGROUND OF THE INVENTION

1. Field of the Invention

The present invention relates to a packing device for a sample inlet formed in an upper cover of a chamber, and more particularly, to a packing device including an air vent, which is formed in the surface of an upper portion of a sample inlet contacting a cap or in the surface of a lower portion of the cap contacting the sample inlet.

2. Description of the Related Art

FIG. 1 is a perspective view illustrating a sample inlet formed in an upper cover of a chamber. Referring to FIG. 1, a chamber 1 used to hybridize reaction materials such as biomolecules include a sample inlet 2 formed in an upper cover of the chamber 1 and a sample is injected through the sample inlet 2. FIG. 2 is a cross sectional view illustrating the movement of a sample solution due to air pressure when a cover is packed into a chamber. Referring to FIG. 2, when the sample is covered by a cover 3, air pressure in the chamber 1 caused by the cover 3 inserted into the sample inlet 2 causes a sample solution 4 to move away from the inlet 2 in which the cover 3 is inserted. That is, hybridization cannot be effectively performed in some areas.

This problem often occurs in chambers used to hybridize a biochip fixed to the bottom of a chamber with a target sample injected through the sample inlet 2. A biochip is formed by affixing to a support a bimolecular probe to be analyzed with high density. The biomolecular probe may be DNA, protein, or the like. By detecting whether the probe is hybridizated with a target material contained in a sample, genetic expression characteristics, genetic defects, protein distribution, reaction characteristics, or the like can be analyzed. Biochips are categorized into DNA chips, protein chips, and the like according to the type of probes used. In addition, biochips are categorized into micro-array chips affixed to solid supports and lab-on-a-chips affixed to micro-channels.

In U.S. Pat. No. 6,739,847 owned by Rechi Precision Co., Ltd. as illustrated in FIG. 3, packing is performed by pressing a valve. In this case, pressure can be absorbed by a spring formed on a cap.

In US 2003/0013184 filed by Tecan Co., as illustrated in FIG. 4, after loading a sample, a general cap is used for packing.

In U.S. Pat. No. 6,432,696 owned by Genomic Solution Co., as illustrated in FIG. 5, after loading a sample, a general cap is used for packing.

As a result of research conducted to solve these problems occurring in the conventional techniques, the inventors of the present invention have found that when a sample inlet of a chamber or a cap has an air vent, these problems can be solved and completed the present invention.

### SUMMARY OF THE INVENTION

The present invention provides a packing device designed such that air pressure caused by the volume of a packing cap inserted into a chamber after the sample is injected into a chamber is not generated inside of the chamber.

According to an aspect of the present invention, there is provided a packing device of a sample inlet formed in an upper cover of a chamber, the packing device comprising an air vent formed in the surface of an upper portion of the sample inlet contacting a cap.

According to another aspect of the present invention, there is provided a packing device of a sample inlet formed in an upper cover of a chamber, the packing device comprising an air vent formed in the surface of a lower portion of a cap contacting the sample inlet.

The chamber may be a reaction camber in which materials react, and preferably, biomolecules are hybridized.

In the packing device, the cap may be separated from the upper cover of the chamber, and preferably formed in a lower surface of a second cover disposed on the upper cover of the chamber. In addition, a side of the second cover may be connected to the upper cover of the chamber by a hinge so that other side of the second cover moves up and down to open or close the sample inlet.

The chamber used to react biomolecules is designed such that air pressure of the upper cover is not generated inside of the chamber when the upper cover is closed. As a result, a spot area can be effectively agitated.

The chamber including a chip and a cover is designed such that air pressure is not generated and the movement of the target sample can be prevented when the chamber is packed. Therefore, effective agitation on the chip can be obtained.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view illustrating a sample inlet formed in an upper cover of a chamber (only a half of the upper cover is illustrated);

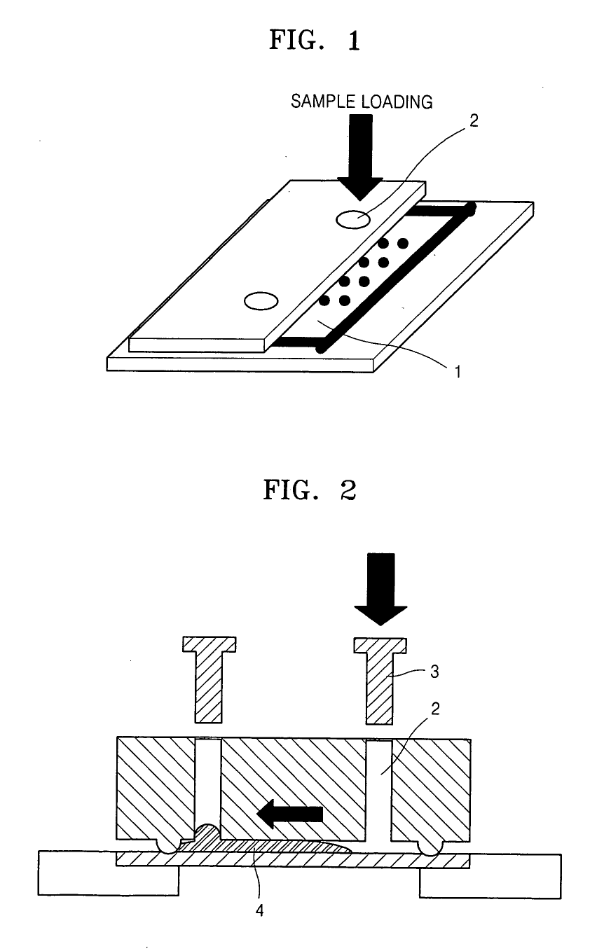

FIG. 2 is a cross sectional view illustrating the movement of a sample solution due to air pressure when a cover is packed into a chamber;

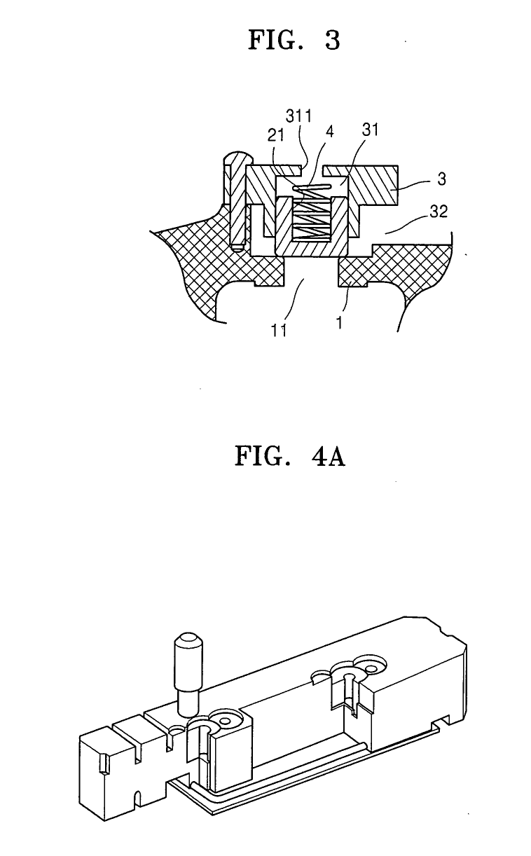

FIG. 3 is a schematic diagram of a conventional packing device obtained from Rechi Precision Co., Ltd;

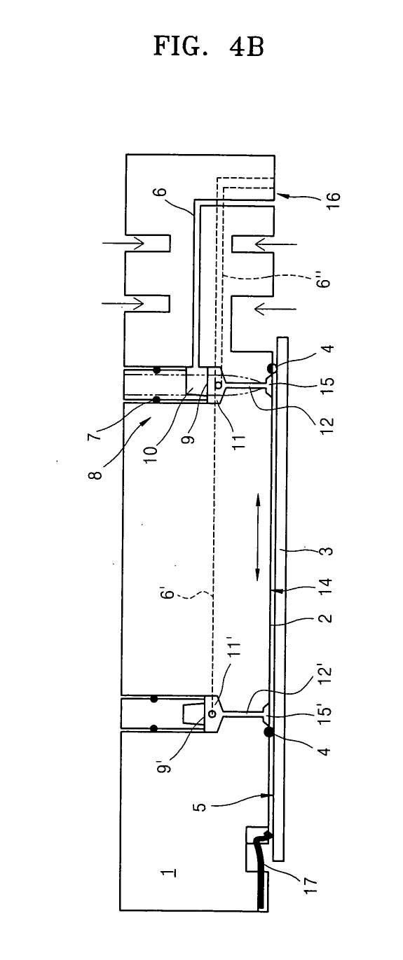

FIG. 4 illustrates a conventional packing device obtained from Tecan Co.;

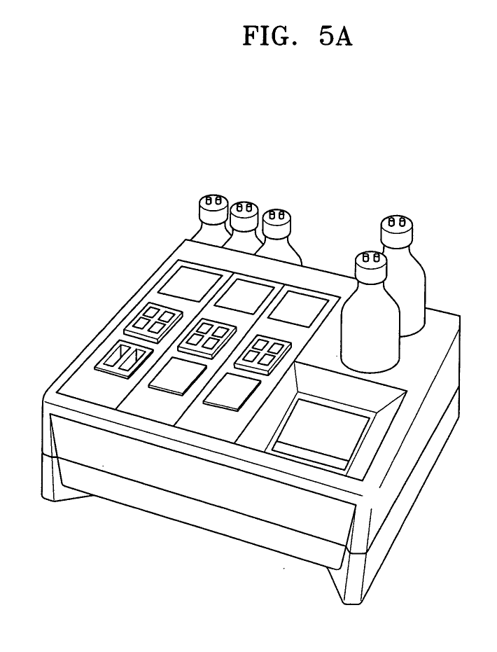

FIG. 5 illustrates a conventional packing device obtained from Genomic solution Co.;

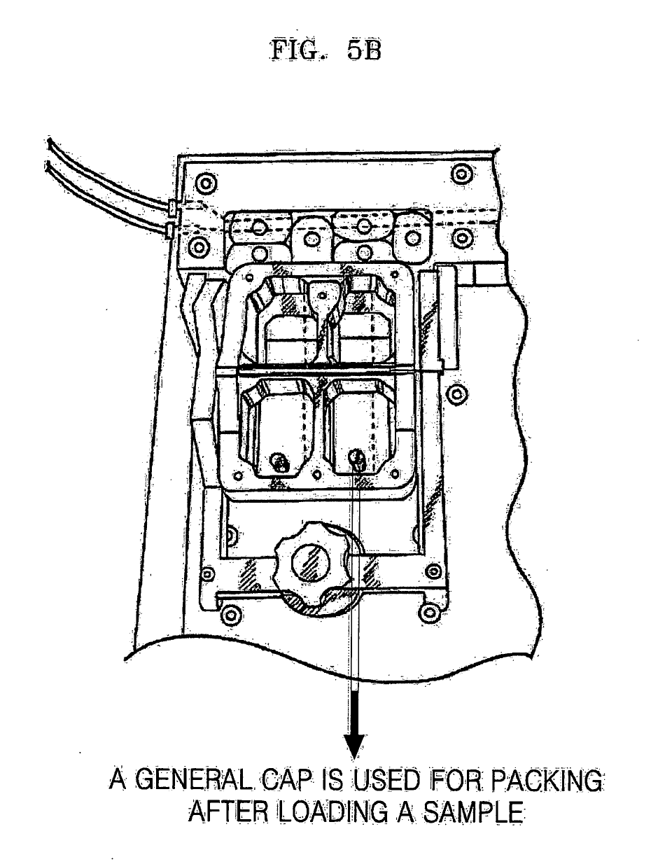

FIG. 6A is a sectional view of a hybridization system including a packing device according to an embodiment of the present invention;

FIG. 6B is a photograph of the hybridization system shown in FIG. 6A;

FIG. 7A is a top perspective view of the hybridization system shown in FIG. 6A;

FIG. 7B is a photograph of a hybridization chamber of the hybridization system shown in FIG. 6A;

FIG. 7C is illustrates an upper cover of the hybridization system shown in FIG. 6A;

FIG. 8A is a top perspective view of a sample inlet and air channels of the hybridization system shown in FIG. 6A;

FIG. 8B is sectional views of a cover and cap of the hybridisation chamber shown in FIG. 6A;

FIG. 8C illustrates a packing device of the hybridization system shown in FIG. 6A;

FIG. 8D illustrates a packing device of the hybridization system shown in FIG. 6A according to another embodiment of the present invention;

FIG. 8E illustrates when a sample is injected into the hybridization system shown in FIG. 6A and when the sample injection is completed; and

FIG. 9 is an image of the hybridization system shown in FIG. 6.

### DETAILED DESCRIPTION OF THE INVENTION

The present invention will now be described more fully with reference to the accompanying drawings, in which exemplary embodiments of the invention are shown.

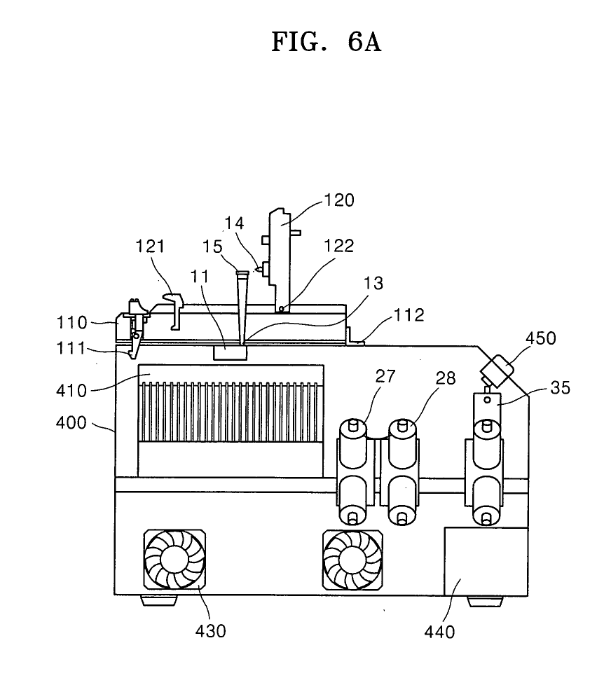

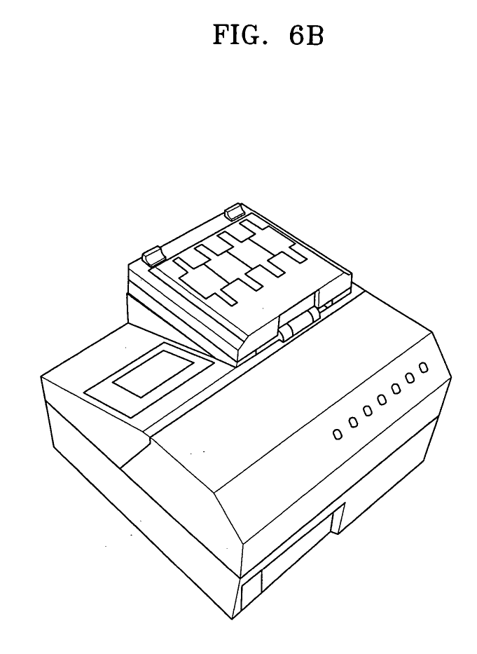

FIG. 6A is a sectional view of a hybridization system including a packing device according to an embodiment of the present invention. Referring to FIG. 6A, the hybridization system uses valves and pumps in a closed system and a first cover 110 connected to a main body 400 by a hinge 112 is held in place or released by a hook 111. A second cover 120 connected to the first cover 110 by a hinge 122 is released from a hook 121. A sample is injected into a hybridization chamber 11 through a sample inlet 13 of the first cover 110 using a micro pipette 15. The second cover 120 includes a cap 14 that covers the sample inlet 13. A heater 410 is disposed below the hybridization chamber 11. The main body 400 includes an agitation device (not shown); a washing/drying device including a first branched valve 27, a second branched valve 36, and a third branched valve 35; a buffer line inlet 450 connected to a buffer container disposed outside the system; an air pan 430 removing heat generated inside the main body 400; and an air pump 440. In addition, an LCD monitor 420 displaying system operations is disposed on a slanted outer surface of the main body 400.

FIG. 6B is a photograph of the hybridization system shown in FIG. 6A. The hybridization system may have a size of 30×30×25 (unit: cm), and can be mass-produced for $7,500 each or less. Further, the hybridization system can interface with PCs, and can be connected to a plurality of HS devices. The hybridization system includes a controller and an LCD panel such that the hybridization system can operate independently. Four test chips can be used at the same time, and the hybridization system can operate at a temperature of 0 to 80° C. In the hybridisation system, the agitation for hybridization is performed using a pump, washing is performed using a fluid flow generated by the pump, air is used for drying.

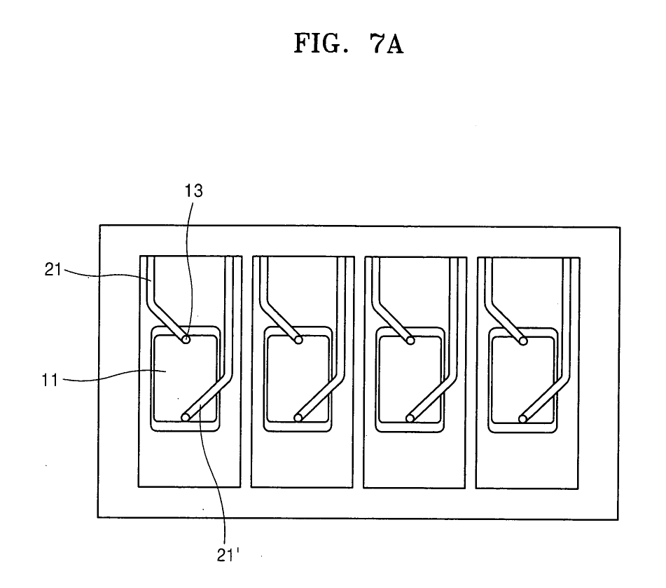

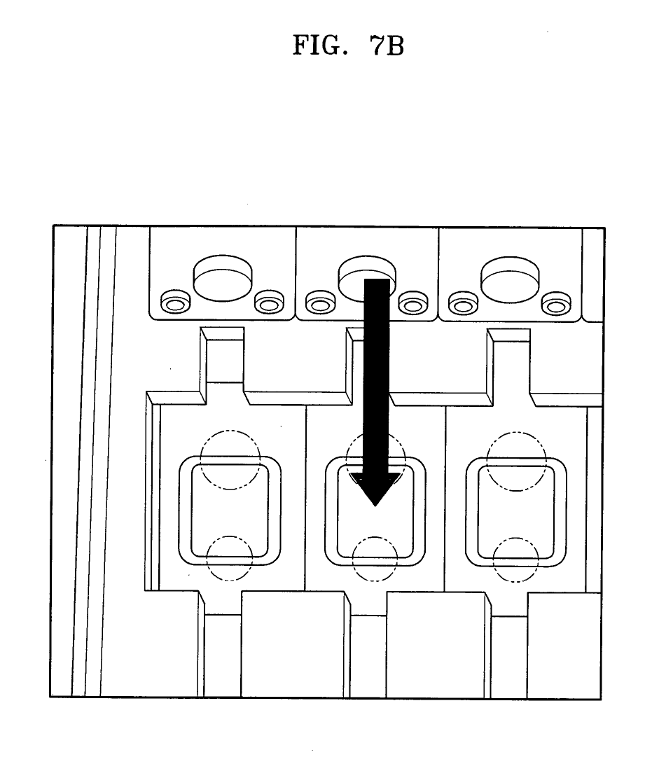

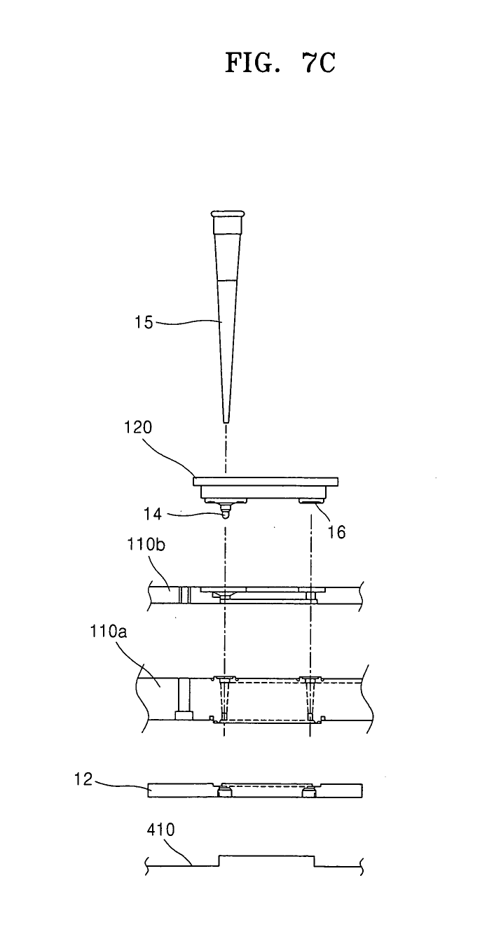

FIG. 7A is a top perspective view of the hybridization system shown in FIG. 6A. Each of the four hybridization chambers 11 is connected to air channels 21 and 21′ via the sample inlet 13 formed at ends of the hybridization chamber 11. The air channels 21 and 21′ extend in the same direction toward a hinge, thereby allowing the opening and closing of a cover. The air channels 21 and 21′ connected to valves and pumps which can be opened or closed form an agitation device (not shown). The air channels 21 and 21′ are connected to side walls of the sample inlet 13, and a channel of a washing/drying device (not shown) is connected to the air channels 21 and 21′ through the branched valves. That is, an integrated channel system in which the sample inlet, the washing/driving device, and the agitation device are connected to the hybridization chamber 11 through a single channel is formed.

FIG. 7B is a photograph of the hybridization chamber 11 of the hybridization system shown in FIG. 6A. When a second cover is moved in the direction indicated by an arrow, the sample inlet 13 is packed by a cap formed on a lower surface of the second cover.

FIG. 7C is illustrates an upper cover of the hybridization system shown in FIG. 6A. Referring to FIG. 7C, a biochip 12 is disposed on the heater 410 and the upper cover is disposed on the biochip 12, thus forming the hybridization chamber 11. The upper cover may include an upper portion 110a composed of rubber and a lower portion 110b composed of transparent acryl. However, the upper portion 110a and the lower portion 110b can be composed of identical materials and integrated with each other. The upper cover is covered by the second cover 120, and a cap 14 that can pack the sample inlet 13 and a bent hole 16 used to obtain smooth diffusion of the sample when the sample is loaded are formed on a lower surface of the second cover 120. The cap 14 faces the bent hole 16. The sample is injected using a pipette 15 after the second cover 120 is opened.

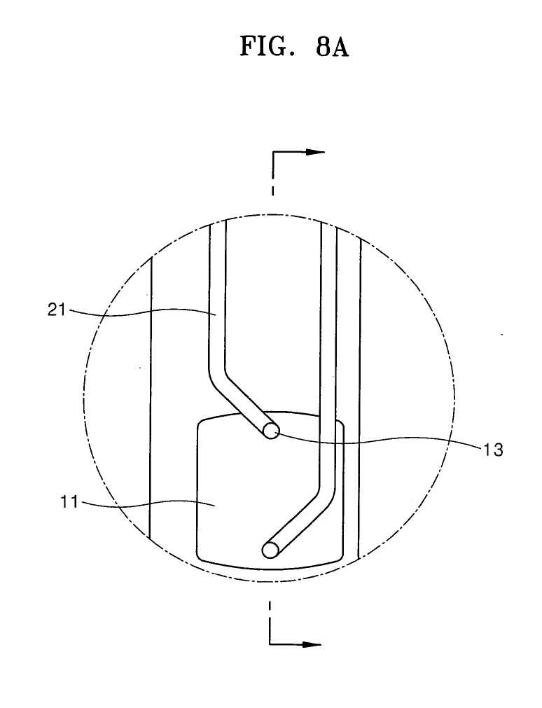

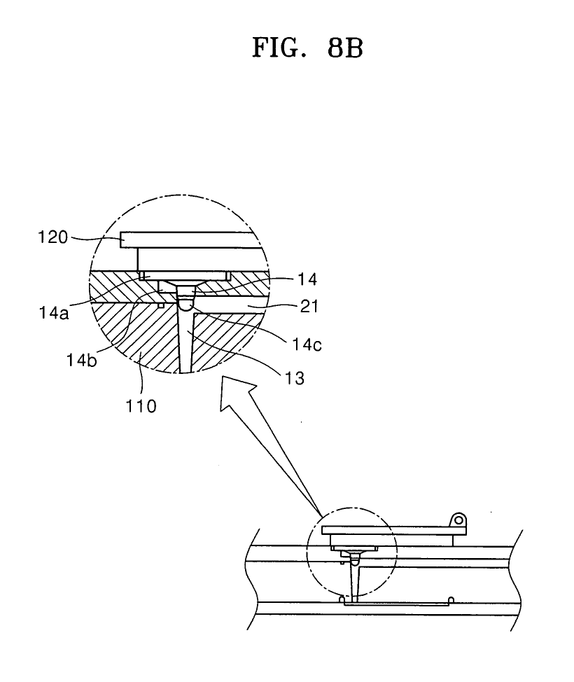

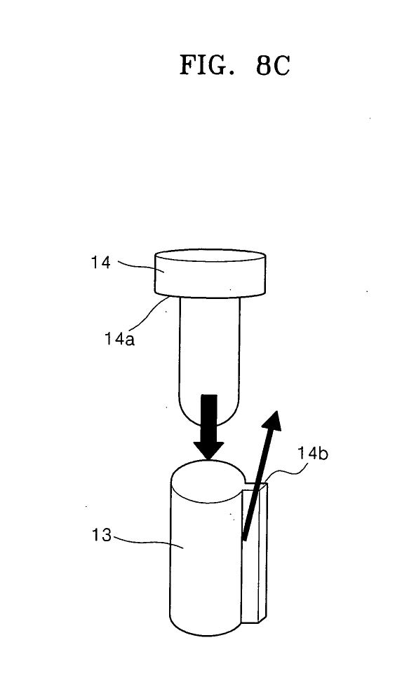

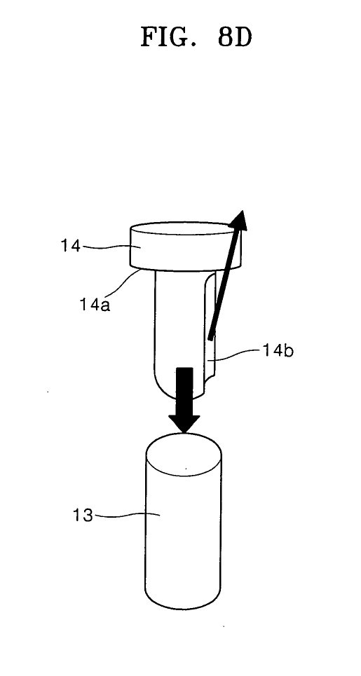

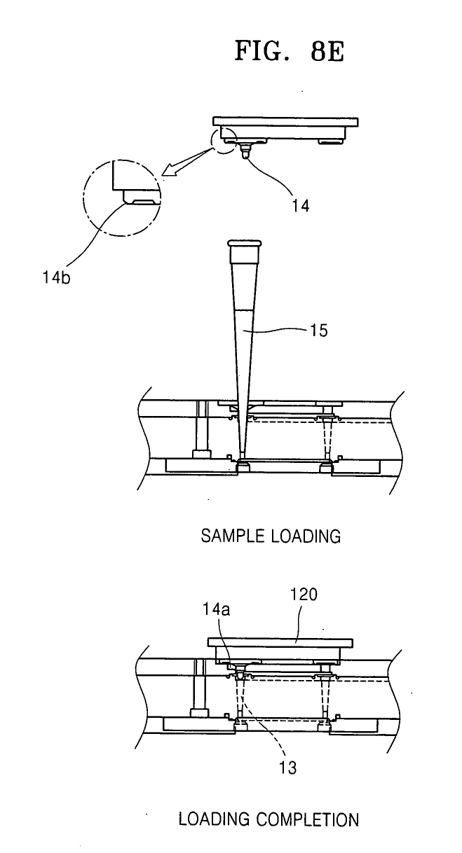

FIG. 8A is a top perspective view of the sample inlet 13 and the air channels 21 and 21′ of the hybridization system shown in FIG. 6A. The air channels 21 and 21′ are connected to ends of the hybridization chamber 11 through the sample inlet 13. The air channels 21 and 21′ extends in the same direction toward the hinge so that the cover can be opened or closed.

FIG. 8B is vertical sectional views of the cover 110 and cap 14 of the hybridisation chamber shown in FIG. 6A. Referring to FIG. 8B, the sample inlet 13 formed in the cover 110 is opened or closed by the cap 14 formed on the lower surface of the second cover 120. The air channel 21 is connected between a side wall of the sample inlet 13 and ends of the hybridization chamber 11. An air vent 14b is formed in the surface of an upper portion of the sample inlet 13. The existence of the air vent 14b allows a decrease in air pressure in the sample inlet 13 when a lower surface 14a¹ of the cap 14 contacts an upper surface of the first cover 110 and a cap top 14c is inserted into the sample inlet 13. Therefore, the movement of the sample solution in the hybridization chamber 11 due to air pressure can be prevented.
¹

[symbol]

FIG. 8C illustrates a packing device of the hybridization system shown in FIG. 6A. Referring to FIG. 8C, the packing device includes the air vent 14b formed on the surface of the upper portion of the sample inlet 13 contacting the cap 14. The air vent 14b has a rectangular shape and protrudes a predetermined distance from a side wall of the sample inlet 13. The air vent 14b allows air pressure, which is generated when the cap 14 is inserted into the sample inlet 13 to be released in the direction indicated by the thin arrow.

FIG. 8D is a view of a packing device of the hybridization system shown in FIG. 6A according to another embodiment of the present invention. Referring to FIG. 8D, the air vent 14b is formed in the surface of the lower portion of the cap 14 contacting the sample inlet 13, which is formed in the upper cover of the chamber. The air vent 14b is a recessed portion with a predetermined length formed in the extended portion of the cap 14. The air vent 14b allows air pressure generated when the cap 14 is inserted into the sample inlet 13 to be released in the direction indicated by the arrow. The sample inlet 13 is sealed by closely contacting the lower surface 14a of an upper portion of the cap 14 with the sample inlet 13. The diameter of the upper portion of the cap 14 is greater than the diameter of the sample inlet 13.

FIG. 8E illustrates when a sample is injected into the hybridization system shown in FIG. 6A and when the sample injection is completed. The second cover 120 is opened and the sample is injected using the pipette 15. After the sample is injected, the sample inlet 13 is sealed by the cap 14 formed on the lower surface of the second cover 120. At this time, the air pressure generated due to the volume of the cap 14 is exhausted via the air vent 14b formed in the surface of the upper portion of the sample inlet 13, and ultimately, the lower surface 14a of the upper portion of the cap 14 with a larger diameter than the sample inlet 13 closely contacts the upper cover of the chamber, thus closing the sample inlet 13.

Hereinafter, the present invention will be described in detail by explaining a preferred embodiment of the invention. The embodiment is provided only to exemplify the present invention, and is not intended to limit the scope of the present invention.

### EXAMPLE

### Test for Sample Movement After Sample Loading

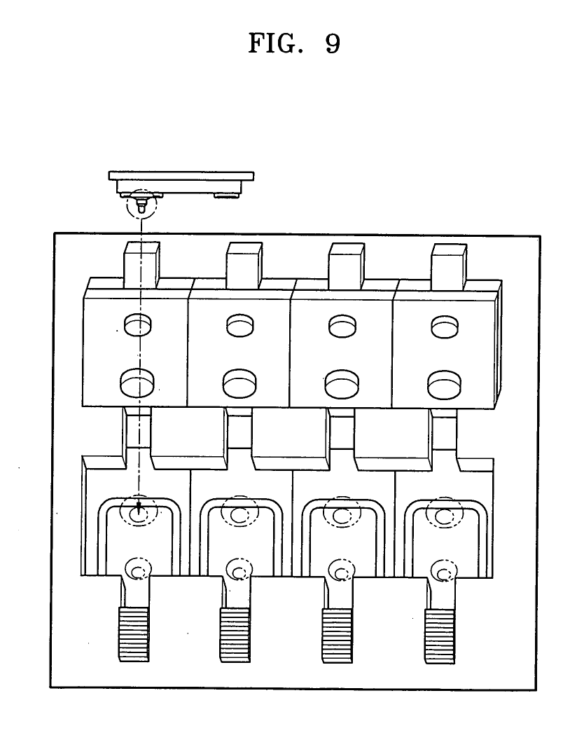

The movement of a target sample in a hybridisation chamber according to an embodiment of the present invention shown in FIG. 9 was measured after loading a sample. 35 μL of a sample solution was injected into a transparent acryl chamber by using a micro pipette, and the chamber was sealed using a cap according to the present invention and the movement of the target sample was measured. As a comparative example, a general cap was used to seal the chamber and the movement of the target sample was measured. As a result, it was observed with the naked eyes that when the chamber was packed using the general cap, the loaded sample was shifted by 3 mm from its initial location. However, when the cap according to the present invention was used, no movement of the sample was observed. That is, while the hybridization chamber had the height of 100 μm and the volume of about 35 μL, a sample inlet has a diameter of about 2,000 μm, which is relatively larger than the volume of the hybridization chamber. Therefore, when the air pressure is not exhausted until the decreased volume due to the injection of the cap reaches about 10 μL, the sample solution in the chamber moves in an opposite direction.

As described above, the movement of the sample solution in a chamber can be prevented by using a structural design in which air pressure generated due to the volume of a packing cap inserted into the sample inlet after the sample is injected into the chamber through the sample inlet to react with reaction materials, such as biomolecules, is not generated inside of the chamber. In addition, in a hybridization chamber of a biochip, the spot area on the biochip can be effectively agitated with the sample solution, thus increasing hybridization efficiency.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A packing device of a sample inlet formed in an upper cover of a chamber, the packing device comprising an air vent formed in the surface of an upper portion of the sample inlet contacting a cap.

2. A packing device of a sample inlet formed in an upper cover of a chamber, the packing device comprising an air vent formed in the surface of a lower portion of a cap contacting the sample inlet.

3. The packing device of claim 1, wherein the chamber is used to hybridize biomolecules.

4. The packing device of claim 1, wherein the cap is formed on a lower surface of a second cover disposed on the upper cover of the chamber.

5. The packing device of claim 2, wherein the chamber is used to hybridize biomolecules.

6. The packing device of claim 2, wherein the cap is formed on a lower surface of a second cover disposed on the upper cover of the chamber.
